(19) United States
(12) Patent Application Publication
(10) Pub. No.: US 2013/0303900 A1
(43) Pub. Date: Nov. 14, 2013

(54) METHOD AND APPARATUS FOR PROCESSING OF STROKE CT SCANS

(71) Applicant: Wieslaw Lucjan Nowinski, Singapore (SG)

(72) Inventor: Wieslaw Lucjan Nowinski, Singapore (SG)

(21) Appl. No.: 13/978,135

(22) PCT Filed: Jan. 30, 2012
(86) PCT No.: PCT/SG2012/000027

§ 371 Date: Jul. 2, 2013

(30) Foreign Application Priority Data
Feb. 1, 2011 (SG) 201100862-0

(51) Int. Cl. A61B 6/00

(52) U.S. Cl. CPC A61B 6/5217
USPC 600/425

(57) ABSTRACT

An automatic technique for stroke identification, localization, quantification and prediction, has the steps of receiving a CT scan, pre-processing it to extract the brain region corresponding to a brain volume of a subject who has suffered a stroke; identifying whether a hemorrhage is present in the brain volume and if so obtaining data characterizing the hemorrhage; otherwise identifying whether an infarct is present and if so obtaining data characterizing it; analyzing the results using a brain atlas; and, using the results of the analysis, obtaining at least one predictive value characterizing a prediction about the subject.

### FIELD OF THE INVENTION

The present invention relates to method for processing a stroke of a brain volume of a subject who has suffered a stroke, to obtain a predictive value indicative of a prognosis for the patient.

### BACKGROUND OF THE INVENTION

A stroke is the rapid loss of brain function due to a disturbance in the blood supply to the brain of subject. It can be due to ischemia (lack of blood flow) caused by a blockage, or a hemorrhage (bleeding within the skull). Ischemic strokes produce cerebral infarctions, in which a region of the brain (an “infarct”) dies due to local lack of oxygen. Ischemic and hemorrhagic phenomena are often mixed, for example because capillaries can be ruptured during reperfusion of an ischemic lesion (that is, when blood flow to an ischemic lesion recommences), triggering a hemorrhage.

Stokes, which have been named the second leading cause of death worldwide, often require rapid diagnosis and appropriate treatment which depends upon the type of stroke, to minimize the rise of permanent neurological damage. Computed tomography (CT) provides the first-line of diagnosis for the evaluation of acute strokes in an emergency situation.

### SUMMARY OF THE INVENTION

In general terms the present invention proposes an automatic technique for stroke identification, localization, quantification and prediction, having the main steps of receiving a CT scan (and optionally other data), pre-processing it to extract the portion corresponding to a brain volume of a subject and to obtain landmarks, identifying whether a hemorrhage is present in the brain volume and if so obtaining data characterizing the hemorrhage; otherwise identifying whether an infarct is present and if so obtaining data characterizing it; analyzing the results using a brain atlas; and using the results of the analysis, obtaining at least one predictive value characterizing a prediction about the subject.

The invention may be expressed as a computerized method, or alternatively as a computer having a processor and a memory device storing software for implementation by the processor to carry out the method. Alternatively, it may be expressed as a computer program product, such as a tangible recording medium, carrying the software.

The predictive value obtained by the method may be employed in further steps of treating the patient. For example, the method enables a quantified prediction of several parameters, such as outcomes (expressed in neurological scales including the Modified Rankin Scale (mRS) or Barthel Index), survival or hospitalization stay. These parameters may be used to select a treatment for a patient, since they will have impact on treatment and healthcare cost (such as hospitalization stay). For instance, patients with a parameter value indicative of a bad predicted outcome could be treated more radically which is usually associated with a higher risk. Patients with a parameter value indicative of a good outcome prediction could take less risky treatment.

The invention may be performed by the computer system automatically (that is, without human involvement except typically to initiate the method) or semi-automatically (that is, with limited human involvement or supervision at one or more of the steps). Automatic implementation is preferable, so that the invention may be performed quickly and even when an expert operator is not available.

### BRIEF DESCRIPTION OF THE FIGURES

An embodiment of the invention will now be described, for the sake of example only, with reference to the following figures, in which:

FIG. 1 is a flow diagram showing the main steps of a method which is an embodiment of the invention;

FIG. 2 shows a pre-processing step of the method of FIG. 1;

FIG. 3 shows a hemorrhage processing step of the method of FIG. 1;

FIG. 4 shows an infarct processing step of the method of FIG. 1; and

FIG. 5 shows an atlas-processing step and a step of forming a prediction, in the method of FIG. 1.

### DETAILED DESCRIPTION OF THE INVENTION

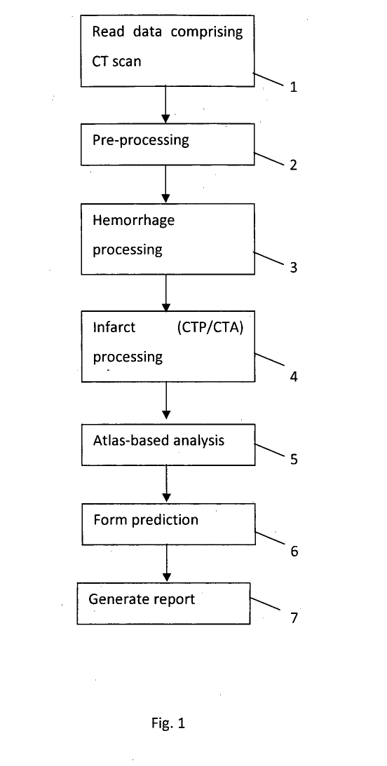

Referring to FIG. 1, an embodiment of the invention is illustrated. As shown in FIG. 1, the method has the overall steps of receiving data 1, pre-processing the data 2, hemorrhage processing 3 (in which the presence of a hemorrhage is first identified, and if this is successful properties of the hemorrhage are obtained), infarct processing 4 (in which the presence of an infarct is first identified, and if this is successful properties of the infarct are obtained), atlas based analysis 5, obtaining a predictive value 6, and generating a report 7. At each of steps 2 to 6 data is generated for inclusion in the report. Note that in other embodiments of the invention the order of the steps may be different. Steps 3 and 4 can be performed in the other order, or even in parallel. An advantage of putting the hemorrhagic stroke processing first is that it may be easier and faster to process, since it may be performed using a single scan. Furthermore, the implementation of step 4 may include a decision which requires information about whether any hemorrhage exists.

In a first step 1 of the method, data is received. The data is an un-enhanced CT scan of (all or part of) the brain volume of a subject. The scan is acquired as a stack of 2D slices. From the image processing standpoint, it is a 3D volume. Some or all of the processing may be performed on the 3-D volume together, rather than slice by slice. For instance, the performance of some of the sub-steps of the method may involve the calculation of the histograms for the whole brain volume (or for each hemisphere separately). Some processing however may be slice-by-slice, or using 2-dimensional sections of the data which are not the same as the acquired slices, or using 3-dimensional subsets of the 3D brain volume. Specific processing may be in any orientation (axial, coronal, sagittal) or any volume/region of interest.

There may be a preliminary step of identifying slices containing strokes, but this is more naturally performed during steps 3 or 4 since slice processing is part of localization of stroke in 3D.

Further data may be received additionally, such as perfusion (CTP) and/or angiography (CTA) scans of the brain volume.

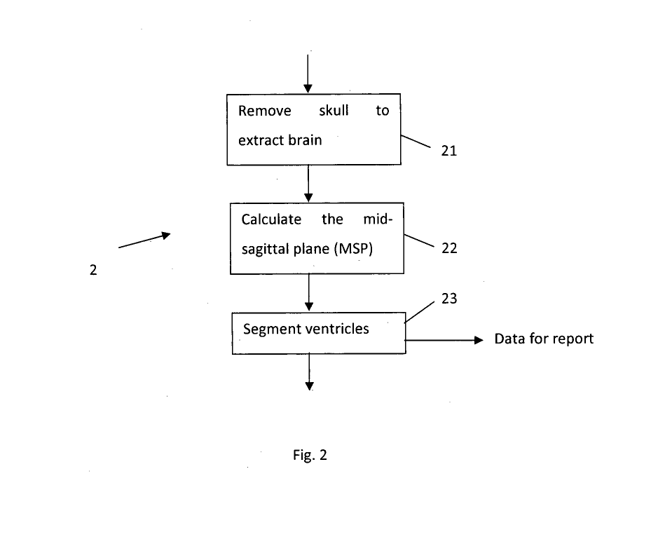

In the second step 2 of the method, the data is pre-processed. The sub-steps of step 2 are illustrated in FIG. 2. The first sub-step 21 is to remove from the images, especially the CT scan, the portions of the image corresponding to the skull and outside the skull, and thereby extracting the portion of the images showing the brain. Suitable techniques are described in [1] and [2]. The second sub-step 22 is the calculation of the mid-sagittal plane (MSP), for instance using the methods of [3] or [4].

The third sub-step 23 is to segment the ventricles within the image, for example using the techniques of [5] and [17].

The results of step 2 are written into the report, and also used in step 3.

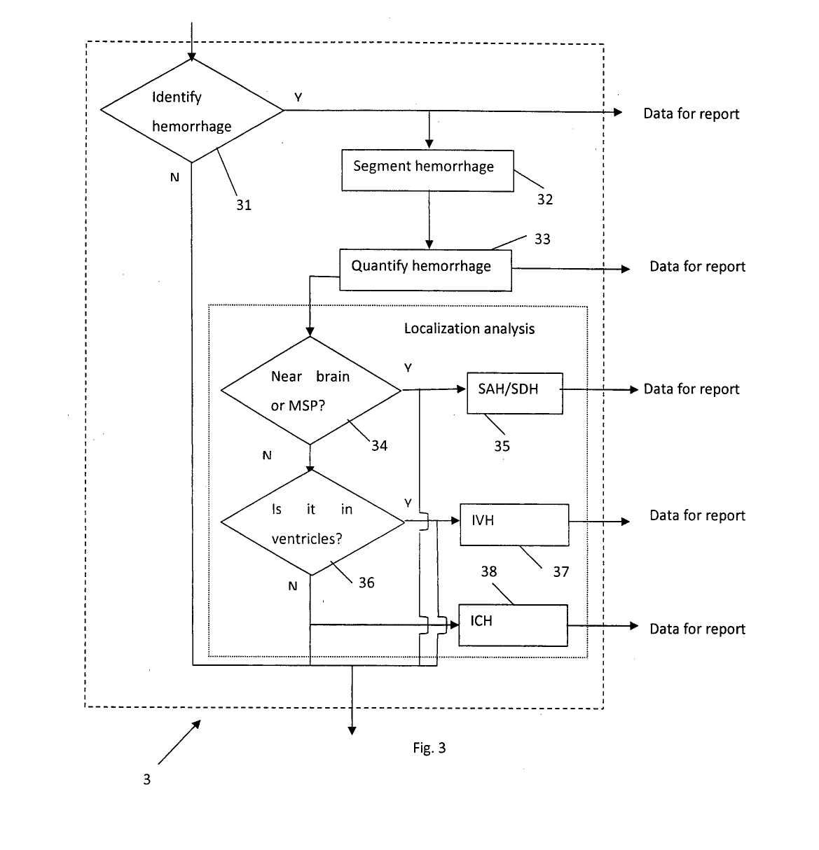

Firstly, in step 3 an attempt is made in sub-step 31 to identify the hemorrhage using a method such as the one in [1]. If no hemorrhage can be identified (“N” in FIG. 3), the method passes to step 4. Alternatively, if a hemorrhage is identified (“Y” in FIG. 3), that information is collected for use in the report, and the method passes to sub-step 32 in which the hemorrhage is segmented by a method such as the one in [1] to obtain location data charactering the location of the hemorrhage (i.e. which portion of the CT image corresponds to hemorrhage). In sub-step 33 the segmented hemorrhage is quantified by measuring its volume.

There is then a localization analysis in which the location of the hemorrhage is characterized, thereby determining the type of hemorrhage: an intracranial hemorrhage (ICH), an intraventricular hemorrhage (IVH) or a subarachnoid/subdural hemorrhage (SAH/SDH). Specifically, in sub-step 34 it is determined whether the hemorrhage is outside the brain in the surrounding volume, meaning that the extracted hemorrhage is neighboring the brain extracted in sub-step 21, or neighboring the mid-sagittal plane (MSP) obtained in sub-step 22, and if so in sub-step 35 it is classified as SAH/SDH. Whether the hemorrhage is “neighboring” can be determined by testing whether one or more criteria are met. Some possible criteria for determining if the hemorrhage neighbors the MSP include: closeness in pixels (e.g. the edge of the hemorrhage closest to the MSP is within a certain number of pixels of the MSP); whether the hemorrhage is within a given radius, or minimum distance (Euclidian or Hausdorff); or whether the hemorrhage overlaps with the MSP. The MSP intersects the third ventricle of the ventricular system, so the overlapping of the hemorrhage with the MSP is only for the cortical areas (i.e., anterior brain and posterior brain), and not within the ventricular system. Another possible criterion (though difficult to implement) is the distance of the centroid of the hemorrhage to the MSP.

In sub-step 36 it is determined if the hemorrhage is in the ventricular system extracted in sub-step 23, and if so in sub-step 37 it is classified as IVH. If both sub-steps 34 and 36 give negative results, then in sub-step 38 the hemorrhage is classified as ICH. The order of steps 34 and 36 may be different in other embodiments of the invention, but it is more natural to perform step 34 first since it is likely to be faster.

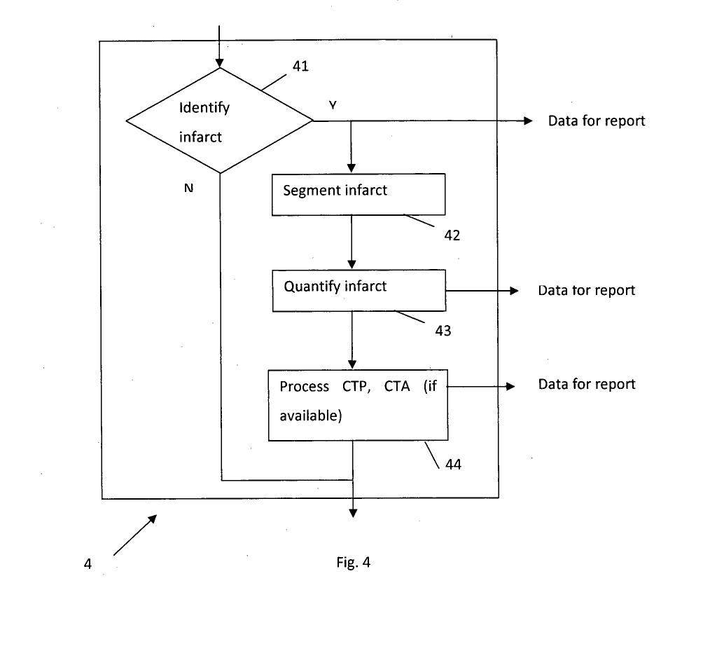

The step 4 of infarct processing is illustrated in FIG. 4. In a first sub-step 41, an attempt is made to identify an infarct, for example by using the method of [7]. If the attempt is unsuccessful the method passes to step 5. If the attempt is successful that result is collected for use in the report, and the method passes to sub-step 42 in which the infarct is segmented (which again may be done using the method of [7]) to obtain location data charactering the location of the infarct (i.e. which portion of the CT image corresponds to infarct), and then to sub-step 43 in which the infarct is quantified by measuring its volume. Alternatively, this volume can be estimated from the identified infarct, e.g. by a method suggested in [7]. In any case, the result is collected for use in the report.

If CTP and CTA scans were received in step 1, these two may be processed, for instance as in [8]. The penumbra can be segmented and quantified by measuring its volume. The mismatch may be calculated. The CTA scan may be analyzed, for instance by employing the method of [8] and visualizing it in three-dimensions. Again, the result is collected for use in the report. In addition, the localization of vascular occlusion may be performed, by employing for instance, method [9], and the result incorporated in the report.

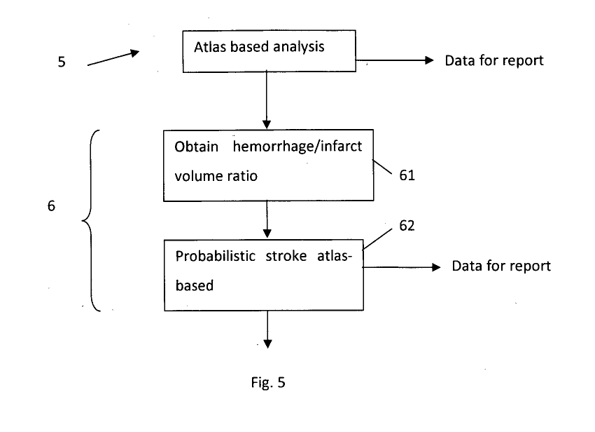

Turning to FIG. 5, the atlas-based analysis step 5 includes mapping a pre-known atlas onto the scan using any of several known methods, such as [10], [11], [12], [13] or [14]. The atlas-based analysis provides underlying neuro-anatomy and blood supply territories. This can be done using a method described in [15]. The localization analysis can be performed for the infarct, penumbra and hemorrhage. The atlas may be mapped onto the scan using any existing method, for instance, those presented in [19]. In particular, a landmark-based method can be employed, such as [14], [19], and landmarks identified as presented in [19]. The atlas-based analysis provides the underlying anatomy for the whole brain as well as any of its region. In particular, the anatomy and blood supply territories can be determined for the infarct (the part of the brain which is lost) and penumbra, meaning the brain's region at risk but able to be salvaged. Moreover the size of the infarct to that of the middle cerebral artery supply (determined from the atlas) is one of the criteria for treatment by thrombosysis [15].

Again, the result of step 5 is collected and used for the report. Already by this stage, enough information has been collected for the report to be a useful tool for diagnosis and treatment, even if the subsequent predictive step 6 is omitted. Note that step 5 is typically performed not in a common space suitable for any individual, but in the image space (patient's space).

The prediction step 6 is performed based on two principles: quantification of hemorrhagic transformation (that is, the amount of the brain which has been transformed by the hemorrhage), and statistical on the basis of a probabilistic stroke atlas. The first principle motivates a sub-step 61 of obtaining the ratio of the hemorrhage volume to the ischemic lesion volume, using the results of sub-steps 33 and 43. This ratio can be used as a predictor. If the ratio is bigger than a given ratio (such as a predetermined value, for instance 30%), it indicates a poor outcome.

The second principle motivates a sub-step 62 of using a probabilistic stroke atlas, for example as discussed in [16], [18], to derive at least one predictive value representing a prediction of concerning the subject. The prediction may be in terms of survival and outcome. For instance, the predictive value may be a prediction of how the subject will score on the Barthel Index at a given time in the future, such as after 30, 60, 180, 360 days. The Barthel index is a scale used to measure the performance of basic activities of daily living. A rating on the Barthel Index is calculated by rating the ability of a patient to perform ten variables describing the activities of daily living and mobility, such that a higher number is associated with a greater likelihood of being able to live at home with a degree of independence following discharge from hospital. Alternatively, the predictive value may be prediction how the subject will score on the modified Rankin scale (mRS) at a given time in the future, such as 7, 30, 90, 180 and 360 days. The modified Rankin scale is a commonly used scale for measuring the degree of disability of dependence in the daily activities of people who have suffered a stroke.

A probabilistic stroke atlas is one of the types of probabilistic atlas proposed in [16] and evaluated in [18]. It is generated by obtaining brain images for many patients who suffered from a stroke earlier. [16] explains the probabilistic stroke atlas particularly in terms of ischemic infarcts, but the method is useful also for hemorrhagic strokes. Each of the images contains damage caused by the stroke (e.g. an ischemic lesion) at a plurality of locations, and each is associated with the value of one or more parameters (Pₙ) characterizing the corresponding patient (e.g. a Barthel Index score or a modified Rankin Scale, for example as measured a certain number of days after the image was captured). To construct this atlas, the brain images and neurological parameters are transformed into a common space defined based on a brain atlas. The probabilistic stroke atlas is then generated in two portions. A first segment (PSA_S) indicates, for each point of the common space, the number of the brain images for which one of the locations was at the corresponding point. The second segment (PSA_Pₙ) exists for each of the parameters, and for each point of the common space, a value indicative of the value of the parameter for those patients for whom one of the locations was at the corresponding point.

In step 62, the probabilistic stroke atlas is mapped onto the subject-specific data, e.g. in the same way as the anatomical atlas is mapped in step 5. If it is determined for a given subject that he or she has stroke damage at a given set of locations, then the probabilistic stroke atlas can be used to produce an estimate of one of the parameters—which, for certain of the parameters means a prediction relating to the patient (for example, if the parameter is a Barthel index score or a modified Rankin scale score for a certain time after the image is captured). This may done for example by extracting, from the portion of the second segment of the probabilistic atlas corresponding to the parameter to be estimated, the values at the locations at which the subject has stroke damage. The distribution of the extracted values gives an estimate for the parameter for the subject, and a measure of the uncertainty in that estimate. The probabilistic stroke atlas can be superimposed onto the patient's scan by using the same methods as those for atlas-scan mapping.

Note that steps 61 and 62 are independent. Step 61, based on clinical observation, is heuristic and qualitative to predict outcome. Step 62 based on the probabilistic stroke atlas is quantitative and can predict not only outcome (in terms of the stroke scales), but also other parameters, such as survival.

As noted above, the localization analysis (sub-steps 34 to 38) classifies the hemorrhage (i.e. ICH, IVH, or SAH/SDH). The embodiment uses this information for the report, but step 6 does not take this information into account. However, in other embodiments of the invention, the hemorrhage classification can be used in step 6 for producing the predictive values and/or in selecting a treatment, e.g. by using a statistical analysis to find correlations between the hemorrhage type and the respective success rates of possible treatments.

The step 62 of using the probabilistic stroke atlas in step 62 can be performed for the whole brain or any of its part defined by the individualized atlases calculated in step 5. For example, over one or more of the blood supply territories obtained in step 5.

The CTP and CTA scans are used to make decision about thrombosysis, which is associated with certain risk. Step 6 facilitates this risk assessment. In principle it would also be possible to generate a probabilistic stroke atlas based on CTP/CTA, though that is not implemented in the present embodiment.

Note that the treatment given to a given subject, and its outcome prediction, depends upon a given situation: a hemorrhage only, ischemia only, or ischemia with hemorrhagic transformation. The invention identifies all three situations.

If both of steps 31 and 42 are negative, then no stroke is detected in the method and steps 5 and 6 are skipped.

### REFERENCES

1. Bhanu Prakash K N, Nowinski W L: Method and system of segmenting CT scan data. U.S. 61/033,105 provisional application filed on 3 Mar. 2008, PCT/SG2009/000079 application filed on 3 Mar. 2009. Publication no. WO/2009/110850 on 11 Sep. 2009.
2. Hu Q, Qian G, Aziz A: Nowinski W L: Brain image segmentation from CT data. U.S. 60/685,175 filed on 27 May 2005; PCT/SG2005/000290 filed on 25 Aug. 2005. Publication no. WO/2006/126970 published on 30 Nov. 2006. SG patent no. 136679 granted on 30 May 2010.
3. Bhanu Prakash K N, Volkov I, Nowinski W L: Locating a mid-sagittal plane, BIL/P//1666/US filed on 2 Apr. 2004. PCT/SG2005/000106 application filed on 1 Apr. 2005. WO 2005/096227 Al published on 13 Oct. 2005. SG200604563-7 filed on 6 Jun. 2006. EP 05722353.9 file on 1 Apr. 2005. SG patent grant no. 200604563-7 granted on 30 Nov. 2007.
4. Hu Q, Nowinski W L: Method and apparatus for determining symmetry in 2D and 3D images. international application number PCT/SG02/00006, 18 Jan. 2002; published international application no: WO03/060827. SG Patent Grant no: 105346 filed on 31 Jul. 2006. 200403898-0. U.S. Pat. No. 7,409,085B2 granted on 5 Aug. 2008.
5. Nowinski W L, Xia Y, Aziz A, Hu Q: Method and apparatus for extracting cerebral ventricular system from images. WO2004/0077359 published on 10 Sep. 2004. SG 200505468-9 filed on 25 Aug. 2005. Provisional application no. U.S. Ser. No. 10/547,441 filed on 25 Aug. 2005. US2006/0239519 Al published 26 Oct. 2006. SG patent grant no. 114360 granted on 30 Nov. 2007.
6. Gupta V, Nowinski W L: Algorithm to segment CSF, white and gray matter in unenhanced computed tomography images. BIL/P/05559/00/SG; SG patent application no. 200904356-3 filed on 23 Jun. 2009.
7. Gupta V, Nowinski W L: Identification, localization and estimation of spatial characteristics of acute infarct region in unenhanced Computed Tomography scans. Submitted on 9 Apr. 2010, SG patent application no. 201003436-1 filed on 14 May 2010
8. Nowinski W L: A method of analysing stroke images. U.S. 60/935,936 provisional application filed on 7 Sep. 2007. Publication no. WO/2009/031973 on 12 Mar. 2009. PCT/SG2008/000303 filed on 18 Aug. 2008. SG patent no. 159792 granted on 21 Dec. 2010.
9. Nowinski W L: Detection and localization of vascular occlusion from angiography data. U.S. 60/842,377 provisional application filed 6 Sep. 2006. US2009/0324052 Al published on 31 Dec. 2009.
10. Nowinski W L, A Thirunavuukarasuu: Method and apparatus for processing medical images. Ref: KRDL/P/0189/1310/US; PCT, EP & US filed PCT/SG00/00185, SG 200303004-6, EP00978194.9 (1344187) & U.S. Ser. No. 10/416,520; published international application no: WO02/43003. SG Patent Grant no: 97358 on 29 Jul. 2005. EP patent no. 1344187, granted on 25 Mar. 2009. U.S. Pat. No. 7,646,898 granted on 12 Jan. 2010.
11. Nowinski W L, Qian G, Bhanu Prakash K N, Hu Q, Aziz A: Fast Talairach Transformation for magnetic resonance neuroimages. Journal of Computer Assisted Tomography 2006; 30(4):629-41.
12. Nowinski W L, Bhanu Prakash K N, Volkau I, Ananthasubramaniam A: Method and apparatus for atlas-assisted interpretation of magnetic resonance diffusion and perfusion images. US60/676,888 provisional application filed on 2 May 2005. PCT/SG2006/000113 and U.S. Ser. No. 11/415,679, 2 May 2006. SG patent no. 137145 granted on 14 May 2010. U.S. Pat. No. 7,783,132 granted on 24 Aug. 2010.
13. Nowinski W L, Beauchamp N: Superimposing brain atlas images and brain images with delineation of infarct and penumbra for stroke diagnosis., U.S. 60/738,071 Provisional application filed on 21 Nov. 2005. US patent application US20090034812 published on 5 Feb. 2009. SG patent no. 141139 granted on 31 Jul. 2008.
14. Volkau I, Bhanu Prakash K N, Ng T T, Gupta V, Nowinski W L: Registering brain images by aligning reference ellipses., U.S. provisional application No. 60/839,711 filed on 24 Aug. 2006. SG patent 148531 granted 30 Sep. 2009.
15. Nowinski W L, Qian G, Bhanu Prakash K N, Thirunavuukarasuu A, Hu Q M, Ivanov N, Parimal A S, Runge V L, Beauchamp N J. Analysis of ischemic stroke MR images by means of brain atlases of anatomy and blood supply territories. Academic Radiology 2006; 13(8)1025-1034.
16. Nowinski W L, Gupta V: “A method for construction and use of a probabilistic atlas for diagnosis and prediction of a medical outcome”, WO/2011/068475.
17. Poh L E, Gupta V, Johnson A, Kazmierski R, Nowinski W L: Automatic segmentation of ventricular CSF from ischemic stroke CT images. Neuroinformatics 2011 DOI: 10.1007/s12021-011-9135-9.
18. Nowinski W L, Gupta V, Qian G Y, Ambrosius W, He J, Kazmierski R: Outcome prediction with a population-based ischemic stroke atlas. Stroke. Proc. International Stroke Conference 2012, New Orleans, USA (in press).
19. Volkau I, Puspitsari F, Nowinski W L: A simple and fast method of 3D registration and statistical landmark localization for sparse multi-modal/time-series neuroimages based on cortex ellipse fitting. The Neuroradiology Journal (in press).

1. A computerized method for detecting stroke, identifying type of stroke, identifying and quantifying any ischemic and hemorrhagic lesions, and obtaining a predictive value characterizing a prognosis for a patient who has suffered a stroke, the method comprising:
(a) receiving a CT scan of a brain region of the subject;
(b) pre-processing the image to identify the portion of it corresponding to the brain, and to obtain brain landmarks;
(c) identifying if the CT scan contains a hemorrhage, and if the determination is positive obtaining location data characterizing the location and volume of the hemorrhage,
(d) identifying if the CT scan contains an infarct, and if the determination is positive obtaining location data characterizing the location and volume of the infarct;
(e) analyzing the location data using a brain atlas and the brain landmarks; and
(f) obtaining at least one predictive value using the results of the analysis of the location data.

2. A method according to claim 1 further comprising:
obtaining a ratio indicative of the volume of the hemorrhage compared to the infarct, the ratio being used in operation (f).

3. A method according to claim 1 in which operation (b) includes extracting the brain from the image.

4. A method according to claim 1 in which operation (b) includes obtaining a mid-sagittal plane for the image.

5. A method according to claim 1 in which operation (b) includes segmenting ventricles in the brain image.

6. A method according to claim 1 in which operation (c) includes characterizing the type of hemorrhage.

7. A method according to claim 6, in which operation (b) comprises obtaining a mid-sagittal plane for the image, and the operation of characterizing the type of hemorrhage comprises determining the position of the hemorrhage in relation to the mid-sagittal plane, and identifying the hemorrhage as a subarachnoid hemorrhage SAH or subdural hemorrhage SDH according to the proximity of the hemorrhage to the brain or mid-sagittal plane.

8. A method according to claim 6, in which operation (b) comprises segmenting ventricles in the brain image, and the operation of characterizing the type of hemorrhage comprises determining the proximity of the hemorrhage to the segmented ventricles, and identifying the hemorrhage as an intra-ventricular hemorrhage (IVH) according to the proximity.

9. A method according to claim 1 in which operation (a) additionally comprises receiving at least one perfusion scan (CTP) scan of the brain of the subject and/or at least one angiography (CTA) scan of the brain of the subject, and operation (d) further comprises analyzing said CTP and/or CTA scan.

10. A method according to claim 1 in which operation (e) includes obtaining neuro-anatomy and blood supply territory information from the brain atlas, said neuro-anatomy and blood supply territory information being used in operation (f).

11. A method according to claim 1 in which operation (f) is performed using a probabilistic stroke atlas, which associates locations of the brain with values of the predictive parameter, the probabilistic stroke atlas having been generated from data relating to other individuals than the subject.

12. A computer system having a processor and a data storage device, the data storage device storing program instructions for implementation by the processor to cause processor to:
(a) pre-process a CT scan of a brain region of the subject to identify the portion of it corresponding to the brain, and to obtain brain landmarks;
(b) identify if the CT scan contains a hemorrhage, and if the determination is positive obtaining location data characterizing the location and volume of the hemorrhage,
(c) identify if the CT scan contains an infarct, and if the determination is positive obtaining location data characterizing the location and volume of the infarct;
(d) analyze the location data using a brain atlas and the brain landmarks; and
(e) obtain at least one predictive value using the results of the analysis of the location data.

13. A computer program product such as a tangible data storage device, storing non-transitory program instructions for implementation by a processor of a computer system to cause the processor to:
(a) pre-process a CT scan of a brain region of the subject to identify the portion of it corresponding to the brain, and to obtain brain landmarks;
(b) identify if the CT scan contains a hemorrhage, and if the determination is positive obtaining location data characterizing the location and volume of the hemorrhage,
(c) identify if the CT scan contains an infarct, and if the determination is positive obtaining location data characterizing the location and volume of the infarct;
(d) analyze the location data using a brain atlas and the brain landmarks; and
(e) obtain at least one predictive value using the results of the analysis of the location data.

14. A method of selecting a treatment for a subject who suffers from a stoke, comprising:
(a) receiving a CT scan of a brain region of the subject;
(b) pre-processing the image to identify the portion of it corresponding to the brain, and to obtain brain landmarks;
(c) identifying if the CT scan contains a hemorrhage, and if the determination is positive obtaining location data characterizing the location and volume of the hemorrhage,
(d) identifying if the CT scan contains an infarct, and if the determination is positive obtaining location data characterizing the location and volume of the infarct;
(e) analyzing the location data using a brain atlas and the brain landmarks;
(f) obtaining at least one predictive value using the results of the analysis of the location data; and
(g) selecting a treatment for the patient using the predictive value.

15. A method of treating a subject who suffers from a stoke, comprising:
(a) receiving a CT scan of a brain region of the subject;
(b) pre-processing the image to identify the portion of it corresponding to the brain, and to obtain brain landmarks;
(c) identifying if the CT scan contains a hemorrhage, and if the determination is positive obtaining location data characterizing the location and volume of the hemorrhage,
(d) identifying if the CT scan contains an infarct, and if the determination is positive obtaining location data characterizing the location and volume of the infarct;
(e) analyzing the location data using a brain atlas and the brain landmarks; and
(f) obtaining at least one predictive value using the results of the analysis of the location data;
(g) selecting a treatment; and
(h) performing the treatment.
